(19) United States
(12) Patent Application Publication
(10) Pub. No.: US 2008/0188670 A1
(43) Pub. Date: Aug. 7, 2008

(54) 1,4-Dithienylbenzene Derivative

(75) Inventors: Yo Shimizu, Osaka (JP); Hirosato Mononobe, Osaka (JP); Kazuma Oikawa, Saitama (JP); Kazuhiko Tsuchiya, Saitama (JP); Junpei Takahashi, Saitama (JP)

Correspondence Address: OBLON, SPIVAK, MCCLELLAND MAIER & NEUSTADT, P.C., 1940 DUKE STREET, ALEXANDRIA, VA, 22314, US

(73) Assignees: NAT. INST. OF ADVANCED INDUSTRIAL SCI. AND TECH., Tokyo (JP); Kanto Kagaku Kabushiki Kaisha, Tokyo (JP)

(21) Appl. No.: 11/661,321

(22) PCT Filed: Feb. 28, 2005
(86) PCT No.: PCT/JP2005/003282

§ 371 Date: Oct. 1, 2007

(30) Foreign Application Priority Data
Aug. 27, 2004 (JP) 2004-248910

(51) Int. Cl. C07D 409/00

(52) U.S. Cl. 549/59

(57) ABSTRACT

Provided are a novel compound that is mesogenic or liquid crystalline and has a high charge mobility, liquid crystal compositions comprising the compound, charge transport materials containing the same, and various elements using the charge transport materials. The novel compound is a 1,4-dithienylbenzene derivatives represented by the following general formula (I):

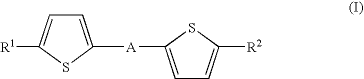

wherein R¹ and R² each independently represent a hydrogen atom or a hydrocarbyl group having 1 to 20 carbon atoms, provided that R¹ and R² are not simultaneously a hydrogen atom; and A represents an optionally substituted benzene ring.

### TECHNICAL FIELD

The present invention relates to novel 1,4-dithienylbenzene derivatives, liquid crystal compositions containing said derivative, charge transport materials containing said derivative, and various elements using said charge transport materials.

### BACKGROUND ART

Organic semiconductors (liquid crystal organic semiconductor) having mesophases are materials characterized by possessing both of the large area homogeneity required for amorphous organic semiconductor materials and the molecular orientation required for crystal organic semiconductor materials. The possibilities of these materials for various applications, such as a photoelectric conversion device and an electroluminescent device, have been explored. The charge transport characteristics and photoconductive behavior of these liquid crystalline organic semiconductors are increasingly attracting attention, inspired by a report disclosing that the semiconductors exhibit a high hole mobility of 5×10⁻³ cm²/Vs which is 1000 times as large as those of amorphous organic semiconductors under the conditions called “a smectic A phase” of phenylbenzothiazole (A) which is one of rod-like liquid crystals (for example, non-Patent Document 1).

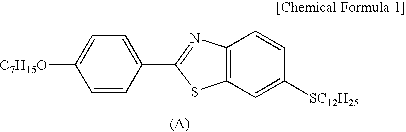

Subsequently, the bipolar charge transport of 1×10⁻² cm²/Vs has been observed in a smectic E phase of 2-phenylnaphthalene (B), as well as in a smectic G phase of dioctylterthiophene (C) (Patent Document 1).

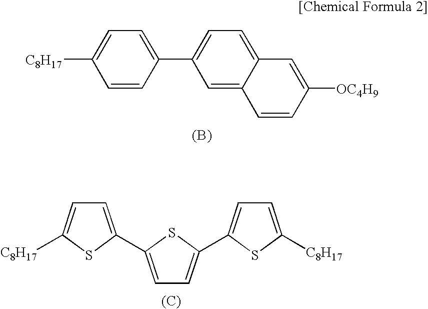

The hole mobility of 6×10⁻² cm²/Vs has been obtained in a smectic B crystal phase of a terthiophene derivative (D) whose molecule symmetry is broken.

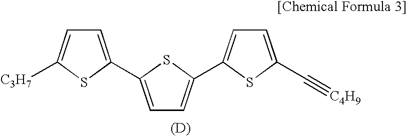

An oxadiazole derivative (E) has been observed to exhibit the electronic conduction of 8×10⁻⁴ cm²/Vs in a phase called “a smectic X phase”.

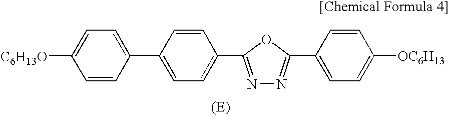

An anthracene derivative and a benzothienobenzothiophene derivative both have been observed to exhibit the hole mobility of 2×10⁻³ cm²/Vs in a smectic C phase, and bipolar charge transport of 2×10⁻³ cm²/Vs in a smectic A phase.

However, the mobility in the conventional rod-like liquid crystals is extremely smaller than 0.1 to 1 cm²/Vs of molecularity crystals, and its high mobility remained the biggest problem.

[Patent Document 1] Japanese Patent Laid-Open No. 2001-233872

[Non-Patent Document 1] Jpn. J. Appl. Phys., 35, 703, 1996.

An object of the present invention is to provide novel compounds that are mesogenic or liquid crystalline and have the high charge mobility, liquid crystal compositions comprising the compound, charge transport materials containing the same, and various elements etc. using the charge transport materials.

### DISCLOSURE OF THE INVENTION

The present inventors have conducted earnest studies based on the aforementioned problems. As a result, the present inventors have found that a compound having a 1,4-dithienylbenzene skeleton and represented by the following general formula (I) is mesogenic and has a high charge mobility, and the compound or liquid crystal compositions containing the compound are useful for various devices or elements as charge transport materials, and the present invention has been thus completed.

Specifically, the present invention relates to 1,4-dithienylbenzene derivatives represented by the following general formula (I):

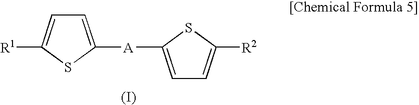

wherein R¹ and R² each independently represent a hydrogen atom or a hydrocarbyl group having 1 to 20 carbon atoms, provided that R¹ and R² are not simultaneously a hydrogen atom; and A represents an optionally substituted benzene ring.

The present invention relates to liquid crystal compositions containing the 1,4-dithienylbenzene derivatives.

The present invention relates to charge transport materials containing the 1,4-dithienylbenzene derivatives or the liquid crystal compositions containing the 1,4-dithienylbenzene derivatives.

Furthermore, the present invention relates to photoelectron conversion devices and electroluminescent devices which use the charge transport materials.

The 1,4-dithienylbenzene derivatives of the present invention are mesogenic and have a high charge mobility. Therefore, the compound or liquid crystal compositions containing the compound can be excellent charge transport materials which are fast and of high-quality, and are useful as materials for various devices such as photoelectron exchange devices or elements.

### BEST MODE FOR CARRYING OUT THE INVENTION

Examples of hydrocarbyl groups represented by R¹ and R² in the general formula (I) and having 1 to 20 carbon atoms include straight-chain or branched saturated or unsaturated hydrocarbyl groups having 1 to 20 carbon atoms, and cyclic saturated or unsaturated hydrocarbyl groups having 3 to 20 carbon atoms. Of these, in view of mesophase formation, those having 4 to 12 carbon atoms are preferable. The straight-chain hydrocarbyl groups in which R¹ and R² are different from each other are preferable to extend a temperature range where the mesophase is developed. When one of R¹ and R² is a hydrogen atom or a hydrocarbyl group having 1 to 3 carbon atoms, it is preferable that the other is a hydrocarbyl group having carbon atoms of 8 or more. Herein, the mesophase is a general term of a phase state which is located in the middle of a crystal phase and liquid phase and has a fixed molecular orientation order. The mesophase means a molecule condensed state which induces actions of liquid crystal phases such as a nematic liquid crystal phase, a smectic liquid crystal phase, and anisotropy plastic crystal (crystal liquid crystal phase), a discotic liquid crystal phase, a cholesteric liquid crystal phase and an optical isotropy liquid crystal phase. Therefore, the mesogenic compound may not represent a liquid crystal phase itself necessarily. The mesogenic compound needs only to represent the action of the liquid crystal phase when mixed with the other compound. Since the mesogenic compound has two advantages of the large area homogeneity of amorphous materials and molecular alignment of crystal materials, the mesogenic compounds are advantageous for device productions.

Examples of the straight-chain saturated hydrocarbyl groups include straight-chain alkyl groups having 1 to 20 carbon atoms such as a methyl group, an ethyl group, a propyl group, a butyl group, a pentyl group, a hexyl group, an octyl group and a dodecyl group. Examples of the straight-chain unsaturated hydrocarbyl groups include straight-chain alkenyl groups having 2 to 20 carbon atoms such as a vinyl group, a 1-propenyl group, a 1-butenyl group, a 1-pentenyl group and a 1-hexenyl group, and straight-chain alkynyl groups having 2 to 20 carbon atoms such as ethynyl, 1-propynyl, 1-butynyl, 1-pentynyl, 1-hexynyl and 1-octynyl.

Examples of the branched saturated hydrocarbyl groups include branched alkyl groups having 3 to 20 carbon atoms such as isopropyl, an isobutyl group, an isopentyl group and an isohexyl group. Examples of the branched unsaturated hydrocarbyl groups include branched alkenyl groups having 3 to 20 carbon atoms such as an isopropenyl group, a 1-isobutenyl group, a 1-isopentenyl group and 1-isohexenyl, and branched alkynyl groups having 3 to 20 carbon atoms such as an isopropynyl group, 1-isobutynyl, 1-isopentynyl and 1-isohexynyl.

Examples of the cyclic saturated hydrocarbyl groups include cycloalkyl groups having 3 to 20 carbon atoms such as a cyclopropyl group, a cyclohexyl group, a cycloheptyl group and a cyclooctyl group. Examples of the cyclic unsaturated hydrocarbyl groups include cycloalkenyl groups such as a 1-cyclopropenyl group, a 1-cyclobutenyl group and 1-cyclohexenyl, or cyclo alkynyl groups having 3 to 20 carbon atoms such as 1-cyclobutynyl and 1-cyclohexynyl.

1 to 4 substituents may exist on the benzene ring represented by A unless they have any effect on the charge mobility. Examples of the substituents include a cyano, a nitro group, lower alkyl groups such as a methyl group, and halogen atoms such as a fluoride atom and a chlorine atom.

The 1,4-dithienylbenzene derivatives represented by the general formula (I) can be produced by a method represented by, for example, the following production example 1.

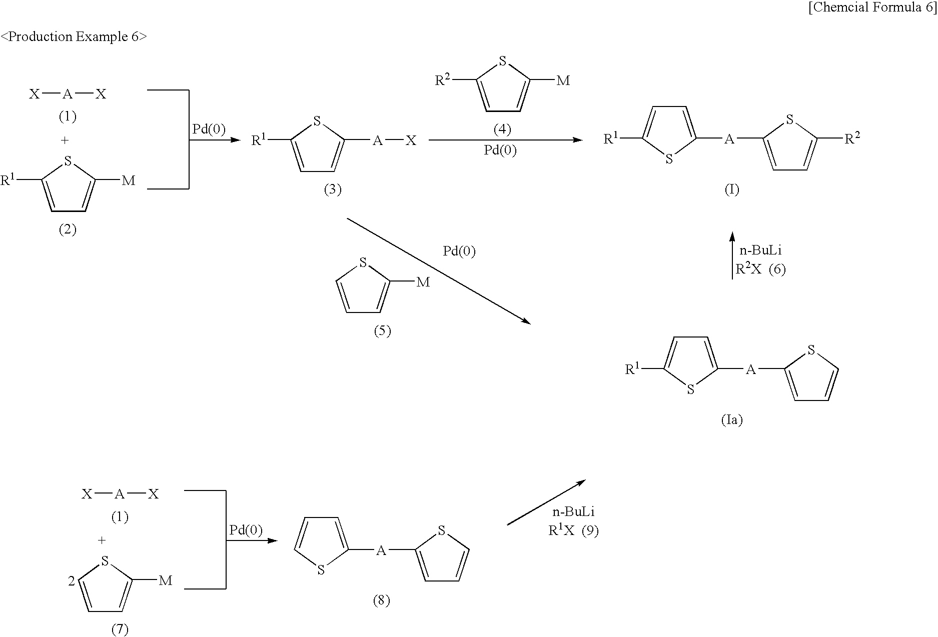

In the above scheme, R¹, R² and A are the same as those described above; X represents B(OR)₂, SnR₃, Br, Cl, I, OTf, MgCl or ZnCl; and M represents B(OR)₂, SnR₃, Br, Cl, I, OTf, MgCl or ZnCl (herein, R represents a hydrogen atom or a lower alkyl group.).

Specifically, a compound (I) of the present invention can be produced by the following methods 1), 2) and 3): 1) a method for reacting a 1,4-disubstituted benzene (1) with thiophene derivatives (2) under a Pd catalyst to produce a thienylbenzene derivative (3) and reacting the thienylbenzene derivatives (3) with thiophene derivatives (4); 2) a method for reacting thiophene derivatives (5) with the compounds (3) to obtain compounds (Ia) of the present invention of which R¹ or R² is a hydrogen atom, and reacting the compounds (Ia) with a compounds (6); and 3) a method for reacting 1,4-disubstituted benzene (1) with thiophene derivatives (7) under a Pd catalyst to produce a compound (8), reacting the compound (8) with compounds (9) to produce compounds (Ia) of the present invention and reacting the compounds (Ia) with compounds (6).

Herein, the reaction of the 1,4-disubstituted benzene (1) and thiophene derivatives (2) or thiophene derivatives (7), and the reaction of the thienylbenzene derivatives (3) and thiophene derivatives (4) or (5) are so-called Suzuki coupling, and can be conducted according to the method described in Chem. Rev., 1995, 95, 2457-2483. As palladium catalysts, tetrakis(triphenylphosphine)palladium (0), tris(dibenzylideneacetone)dipalladium (0), tris(dibenzylideneacetone)dipalladium-chloroform adduct, palladium acetate (II), dichlorobis(triphenylphosphine palladium) (II), dichlorobis(tri-o-triphenylphosphine)palladium (II), dichlorobis(tricyclohexylphosphine)palladium (II), [1,1′-bis(diphenylphosphino)ferrocene]dichloropalladium (II), dichloro[1,2-bis(diphenylphosphino)ethane]palladium(II), and dichloro[1,4-bis(diphenylphosphino)butane]palladium (II). Of these, it is preferable to use the tetrakis(triphenylphosphine)palladium (0) since it is inexpensive and highly active.

The 1,4-disubstituted benzene (1) and thiophene derivatives (2) are preferably mixed at a 0.5 to 1.0 equivalent ratio of (2) to (1). The 1,4-disubstituted benzene (1) and thiophene derivatives (7) are preferably mixed at a 2.0 to 2.2 equivalent ratio of (7) to (1). The thienylbenzene derivatives (3) and thiophene derivatives (4) are preferably mixed at a 1.0 to 1.1 equivalent ratio of (4) to (3). The thienylbenzene derivatives (3) and thiophene derivatives (5) are preferably mixed at a 1.0 to 1.1 equivalent ratio of (5) to (3).

The palladium catalyst is preferably used within the range of 0.01 to 0.20 equivalent weight, and more preferably 0.03 to 0.10 equivalent weight since workup and purification after the reaction are simpler. As the reaction solvent, the mixed solvent of the organic solvent and water is preferable. Ethylene glycol dimethyl ether, N,N-dimethylformamide, tetrahydrofuran, 1,4-dioxane, toluene, benzene and acetone are used as the organic solvent. For each solvent, water is used in an amount of 0.05 to 1.5 times, and more preferably in an amount of 0.3 to 1.2 times to the organic solvent. Although a base used in the reaction may be sodium carbonate, sodium bicarbonate, triethyl amine, triisopropylamine, sodium ethoxide, cesium carbonate, potassium phosphate, sodium hydroxide, potassium hydroxide, barium hydroxide, tert-butoxy potassium or the like, sodium carbonate is preferable since a weaker base provides good results. When the reaction is adversely influenced by steric hindrance, barium hydroxide or potassium phosphate is preferable. The base is used in a 1.0 to 3.0 equivalent weight, and more preferably 1.8 to 2.2 equivalent weight.

The reaction temperature varies depending on the kinds of the reaction substrate, palladium catalyst, reaction solvent and base, but the reaction may occur within the range of 50 to 150° C. If they are appropriately combined, it is most preferably within the range of 65 to 120° C. The reaction time varies depending on the kinds of the reaction substrate, palladium catalyst, reaction solvent and base as the reaction temperature, but it may be preferably within the range of 1.0 to 10 hours. It is preferably within the range of 2.0 to 5.0 hours if they are appropriately combined.

Also, the reaction to produce the compound (Ia) from the compound (8) and the reaction to produce the compound (I) from the compound (Ia) can be conducted by anionizing the compounds (8) and (Ia) with the anionizing agent and by reacting the resulting anions with R¹X and R²X, respectively.

As the kind of the anionizing agent, aryl lithium such as phenyl lithium, alkaline metal such as sodium metal or lithium metal, lithium amide, lithium dialkyl amide having 2 to 20 carbon atoms such as lithium dimethyl amide or lithium dipropyl amide, and lithium diaryl amide having 12 to 30 carbon atoms including lithium diphenyl amide are used in addition to alkyl lithium having 1 to 20 carbon atoms such as methyl lithium or n-butyl lithium. Since it is well reactive and simple to handle, alkyl lithium or aryl lithium is suitably used, and n-butyl lithium is more suitably used.

The anionizing agent is used at a 1.9 to 2.5 equivalent ratio thereof to the compound (8), and suitably, for example, at a 2.0 to 2.2 equivalent ratio, and it is also used at a 0.9 to 1.2 equivalent ratio thereof to the compound (Ia), and suitably, for example, at a 1.0 to 1.1 equivalent ratio.

For a substrate with a very slow anionization speed or with a very low solubility, any proper quantity of an additive such as N,N,N′,N′-tetramethylethylenediamine (TMEDA), 1,3-dimethylimidazolidin-2-one (DMI) or the like may be added to accelerate its anionization speed.

The reaction temperature for anionization is not particularly limited as long as the anionizing agent hardly decomposes, and it is preferably within the range of −10 to 80° C., and more preferably within the range of −5 to 50° C. since the reaction is easily conducted in those ranges. The reaction time for anionization is preferably 0.5 hours to 10 hours, and more preferably 2 hours to 5 hours.

The solvent used for the anionization is preferably an organic solvent. Hydrocarbons having 5 to 20 carbon atoms such as hexane and pentane, aromatic hydrocarbons having 6 to 20 carbon atoms such as benzene and toluene, and ethers having 4 to 10 carbon atoms such as diethyl ether and tetrahydrofuran are preferable. Ethers are preferable since they are well reactive. Although the amount of the solvent to be used depends on the solubility and reactivity of the substrate, preferably it is generally 3 to 10 mL per 1 mmol of the substrate.

R¹X or R²X is used at a 1.9 to 2.5 equivalent ratio thereof to the compound (8), and suitably, for example, at a 2.0 to 2.2 equivalent ratio, and also used at a 0.9 to 1.2 equivalent ratio thereof to the compound (Ia), and suitably, for example, at a 1.0 to 1.1 equivalent ratio.

The reaction of the compound (8) with R¹X, and the temperature for the reaction of the compound (Ia) with R²X are preferably −80° C. to 50° C., and more preferably −50 to 30° C. The time for the reaction of the compound (8) with R¹X, and the time for the reaction of the compound (Ia) with R²X are preferably 3 hours to 24 hours, and more preferably 5 hours to 15 hours.

The compound (Ib) in which R¹ and R² are the same hydrocarbyl group can be also prepared, for example, by the following production example 2.

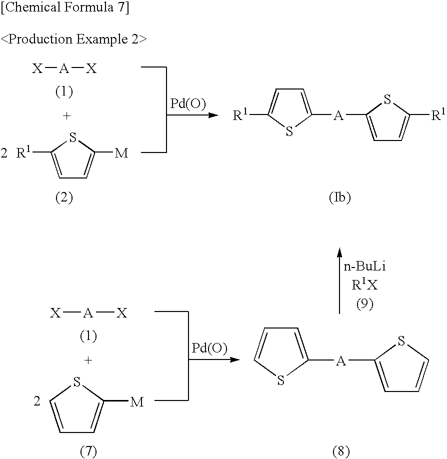

In the above scheme, R¹, A, X and M are the same as those described above.

Specifically, the compound (Ib) can be prepared by 1) a method for reacting the 1,4-disubstituted benzene (1) with the thiophene derivative (2) under the Pd catalyst, and 2) a method for obtaining a compound (8) from the 1,4-disubstituted benzene (1) and the thiophene derivative (7) and reacting the compound (8) with a compound (9).

Compound (Ic) and (Id) in which R¹ and R² are unsaturated hydrocarbyl can be also prepared, for example, by the following producing example 3.

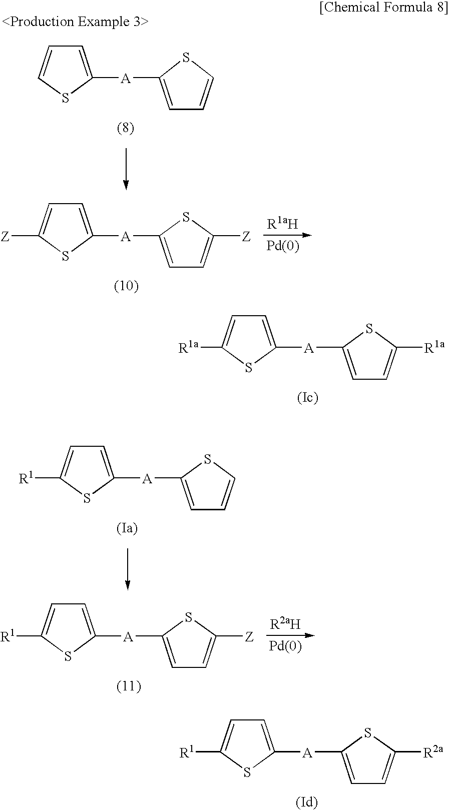

In the above scheme, R^{1a} and R^{2a} represent a chain unsaturated hydrocarbyl group having 4 to 20 carbon atoms, and Z represents Li, B(OR)₂, SnR₃, Br, Cl, I, OTf, MgCl or ZnCl, wherein R represents a hydrogen atom or a lower alkyl group, and R¹ and A are the same as those described above.

Specifically, compounds (Ic) or compounds (Id) having an unsaturated hydrocarbyl group can be obtained by first anionizing the compounds (8) or the compounds (Ia) using a known method, for example, with an anionizing agent such as n-butyllithium, then converting the resulting anion into compounds (10) or compounds (11) with tributylstannyl chloride, iodine, bromine, trimethoxy borate or the like, and finally carrying out the Heck reaction where the compounds (10) or (11) is cross coupled using an end olefin with a PdCl₂ catalyst to produce a substituted olefin, or the Sonogashira reaction where a Pd (0) catalyst, copper iodide and an amine are added to the compounds (10) or (11), and an end acetylene is further added to cause cross coupling. The Heck reaction can be conducted according to the method described in R. F. Heck, “Palladium Reagents in Organic Synthesis,” Academic Press, 1985, Chap. 6. The Sonogashira reaction can be conducted according to the method described in K. Sonogashira et al., T L, 50, 4467, 1975.

The 1,4-dithienylbenzene derivatives of the present invention thus obtained are mesogenic compounds, and have large area homogeneity, molecular alignment and mobility (see Examples). Referring to the charge mobility of the 1,4-dithienylbenzene derivatives, the hole or electron mobility is 1×10⁻³ cm²/Vs or more, and the 1,4-dithienylbenzene derivatives have a much higher hole mobility than dioctylterthiophene, and thereby the 1,4-dithienylbenzene derivatives are useful as charge transport materials for using for a semiconductor layer of a photoelectric conversion device and an electroluminescent device.

The 1,4-dithienylbenzene derivatives of the present invention can be incorporated into liquid crystal compositions containing one or more species thereof, and a different liquid crystalline or non-liquid crystalline compound, a synthetic organic polymer and the like as long as the 1,4-dithienylbenzene derivatives does not suppress large area homogeneity, molecular alignment, hole and/or electron mobility as described above. For example, the liquid crystal compositions containing 90 wt % to 10 wt % of the 1,4-dithienylbenzene derivatives of the present invention can be used. The composition are also useful as the charge transport materials. Herein, as the other liquid crystalline compounds and non-liquid crystalline compounds, any known one can be used. Thermoplastic polymers, thermosetting polymers, engineering plastics, conductive polymers or the like can be used as synthetic organic polymers. Various additives may be further contained in the liquid crystal compositions, and examples of the additives include a plasticizer, a colorant and a dopant. A reinforcing material such as a glass fiber, a carbon fiber and a boron fiber may be further included in the liquid crystal compositions.

The charge transport materials comprising the 1,4-dithienylbenzene derivatives of the present invention or the liquid crystalline composition containing the same has a charge mobility where its hole or electron mobility is preferably 1×10⁻³ cm²/Vs or more. The charge transport materials used for a semiconductor layer of the photoelectric conversion device or electroluminescent device, in view of the high-speed response and higher efficiency of the device, preferably has a high hole or electron mobility, preferably of 1×10⁻² cm²/Vs or more.

The charge transport materials can be used as the material of various devices or elements. For example, the charge transport materials can be used for electroluminescence elements, photoconductors, thin film transistors, an optical sensors, temperature sensors, image display elements, optical recording elements, photoelectron conversion devices, electroluminescent devices or the like. Since the charge transport materials have high charge mobility, the charge transport materials are preferably used particularly for the optical sensor. Since the charge transport materials have excellent charge transport property, the charge transport materials are preferably used for the electroluminescence elements. Since the charge transport materials have orientation property, photoconductivity and self luminescence, the charge transport materials are preferably used for the image display elements. Examples of the photoelectron conversion devices and electroluminescent devices comprising the charge transport materials of the present invention include devices having layer composed of the charge transport materials of the present invention, which can be exemplified by a glass substrate, an ITO (indium tin oxide) electrode, a liquid crystal alignment layer, a device having a combination thereof or the like.

### EXAMPLES

The present invention will be described below based on Examples, but is not limited to the following Examples.

### Example 1

### 1,4-Bis(5′-octyl-2′-thienyl)-benzene (8TPT8)

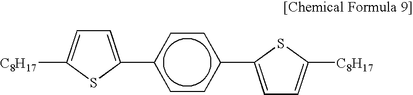

(1) Synthesis of 2-octylthiophene as Intermediate

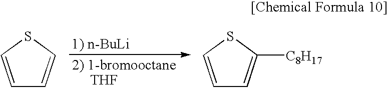

After adding an n-butyllithium/hexane solution (0.3565 mol) to a tetrahydrofuran solution of thiophene (0.3565 mol) cooled to −70° C. and reacting them at room temperature for 3 hours, the obtained solution was cooled to −60° C. again. The 1-bromooctane (0.3565 mol) was dropped into the solution, and 1-bromooctane was reacted with the solution at room temperature for 15 hours. After removing the solvent, 300 mL of water was added into a reaction vessel ice-cooled, and the solution was extracted with 300 mL of diethyl ether. The aqueous layer was re-extracted with 100 mL of diethyl ether, and the aqueous layer and the organic layer were neutralized and washed with saturated solution of sodium chloride. The organic layer was dried over sodium sulfate, filtered, concentrated, dried under reduced pressure, and distilled under reduced pressure to obtain 2-octylthiophene (transparent and colorless liquid, 0.2300 mol). Yield: 65%.

(2) Synthesis of 2-octyl-5-tributylstannyl-thiophene as Intermediate

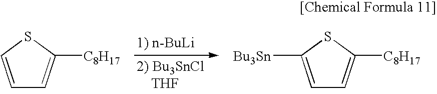

After adding an n-butyllithium/hexane solution (15.279 mol) to a tetrahydrofuran solution of 2-octyl thiophene (15.279 mmol) cooled to −75° C. and stirring them at room temperature for 3 hours, the obtained solution was cooled to −75° C. again. Tributyl stannyl chloride (15.279 mmol) was added to the solution, and the obtained solution was stirred at room temperature for 15 hours. After removing the solvent under reduced pressure, 50 mL of water was added to the reaction vessel water-cooled, and the solution was extracted with 150 mL of diethyl ether. Then, the extracted solution was water-washed, and the organic layer was dried over sodium sulfate, filtered, concentrated and dried under reduced pressure to obtain 2-octyl-5-tributyl stannyl-thiophene (13.803 mmol). Yield: 90%.

(3) Synthesis of 1,4-bis(5′-octyl-2′-thienyl)-benzene

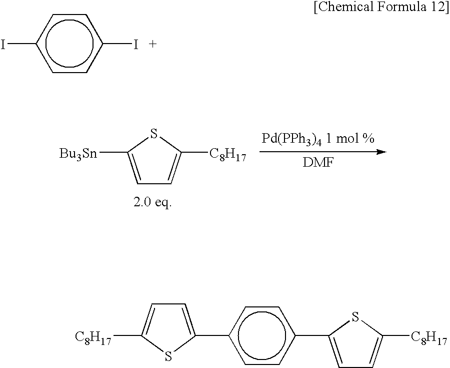

After a DMF solution of 1,4-diiodobenzene (6.547 mmol), 2-octyl-5-tributylstannyl-thiophene (13.095 mmol) and tetrakis(triphenylphosphine palladium) (0) (0.065 mmol) was heated at 85° C. for 4 hours, the DMF solution was ice-cooled and water was then added thereto. The DMF solution was then extracted with 200 mL of diethyl ether, and the extracted solution was washed with saturated solution of sodium chloride and then distilled water. The organic layer was dried over sodium sulfate, filtered, concentrated and dried under reduced pressure to obtain 1,4-bis(5′-octyl-2′-thienyl)-benzene (3.942 mmol). Yield: 60%. This crude product was purified by column chromatography, was recrystalized and was then purified by sublimation.

¹H-NMR (CDCl₃, Me₄Si) δ:

0.88 (t, J=7.1 Hz, 6H), 1.28-1.39 (m, 20H), 1.70 (m, 4H), 2.81 (t, J=7.1 Hz, 4H), 6.73 (d, J=3.4 Hz, 2H), 7.12 (d, J=3.4 Hz, 2H), 7.53 (s, 4H).

The analysis results confirmed that the obtained compound was the title compound. UV-Vis spectrum; λₘₐₓ=342 nm (loge4.54)

### (4) Liquid Crystal Temperature Range

Differential scanning calorimetry (DSC) and polarization microscopy demonstrate that 8TPT8 transits from the isotropic phase to a mesophase M1 having a high orientation order at 145° C., to a mesophase M2 at 87° C., further to another mesophase M3 at 71° C., and to a crystal phase at 47° C.

### (5) Charge Transport Characteristics

The charge transport characteristics of the liquid crystalline substance were measured by the Time-of-flight (TOF) method. Referring to an ITO sandwich cell used for measuring, there was used a cell of which both positive and negative electrodes were an ITO electrode; a distance between the electrodes was 15.9 μm; and an electrode area was 0.25 cm². The liquid crystalline substance was encapsulated in the cell under a condition of a temperature of 155° C., and the cell was used as a TOF measurement sample cell. The measurement was conducted at an irradiation wavelength of 337 nm at 120° C., 75° C. and 60° C.

The charge transport of the holes took place in the Ml phase (120° C.), and the charge mobility did not depend on field intensity. The value of hole mobility was 3×10² cm²/Vs. In the M2 phase (75° C.), the value of the hole mobility of 7×10⁻² cm²/Vs was obtained. Furthermore, in the M3 phase (60° C.), the hole mobility having a very high value of 1×10⁻¹ cm²/Vs was obtained.

### Comparative Example 1

As a comparison compound (liquid crystalline substance), dioctylterthiophene (8TTT8) represented by the following formula was used.

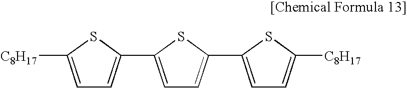

The 8TTT8 was encapsulated under a condition of a temperature of 100° C. in an ITO cell of which a distance between electrodes is 16.3 μm and an electrode area is 0.25 cm², and the cell was used as a TOF measurement sample cell. The measurement was conducted at an irradiation wavelength of 337 nm at 87° C., 80° C. and 70° C. At 87° C. (smectic C phase), the hole mobility was 8.6×10⁻⁴ cm²/Vs. At 80° C. (smectic F phase) , the hole mobility of 2.3×10⁻³ cm²/Vs was obtained. Furthermore, at 70° C. (smectic G phase), the value of the hole mobility of 1.6×10⁻² cm²/Vs was obtained. Each of the hole mobilities were lower than that of Example 1.

### Example 2

### 1-(5′-Butyl-2′-thienyl)-4-(5′-octyl-2′-thienyl)-benzene (8TPT4)

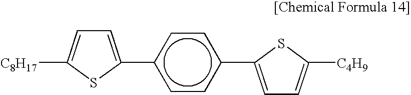

(1) Synthesis of 2-octyl-5-borondimethoxidethiophene as Intermediate

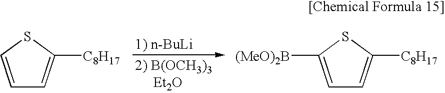

After adding an n-butyllithium/hexane solution (0.165 mol) to a diethyl ether solution of 2-octyl thiophene (0.165 mol) cooled to −75° C. and stirring them at room temperature for 3 hours, the obtained solution was cooled to −75° C. again. Trimethyl borate (0.165 mol) was added to the solution, and the obtained solution was stirred at room temperature for 20 hours. The solvent was removed under reduced pressure to obtain 2-octyl-5-borondimethoxidethiophene (0.140 mol). Yield: 85%.

(2) Synthesis of 1-bromo-4-(5′-octyl-2′-thienyl)benzene as Intermediate

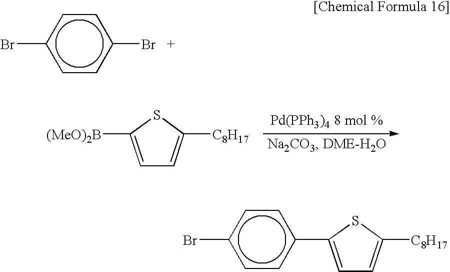

After a suspension of 1,4-dibromobenzene (44.96 mmol), 2-octyl-5-borondimethoxidethiophene (22.48 mmol), tetrakis(triphenylphosphine palladium) (0) (3.597 mmol), sodium carbonate (44.96 mmol), 106 mL of ethylene glycol dimethyl ether and 33 mL of water was heated at 80° C. for 4.5 hours, the suspension was ice-cooled and water was added thereto. Next, the suspension was extracted with 300 mL of chloroform, and the extract solution was washed with saturated solution of sodium chloride and then distilled water. The organic layer was dried over sodium sulfate, filtered, concentrated and dried under reduced pressure to obtain a crude product. This crude product was purified by column chromatography and recrystalized to obtain 1-bromo-4-(5′-octyl-2′-thienyl)benzene (10.24 mmol). Yield: 46%.

(3) Synthesis of 2-butylthiophene as Intermediate

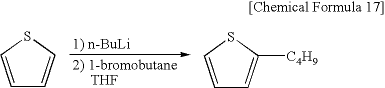

After adding an n-butyllithium/hexane solution (0.178 mol) to a tetrahydrofuran solution of thiophene (0.178 mol) cooled to −70° C. and reacting them at room temperature for 2.5 hours, the obtained solution was cooled to −60° C. again. 1-Bromobutane (0.178 mol) was dropped into the solution to react the obtained solution at room temperature for 15 hours. After removing the solvent, 60 mL of water was added into a reaction vessel ice-cooled, and the solution was extracted with 100 mL of diethyl ether. The aqueous layer was re-extracted with 100 mL of diethyl ether, and the aqueous layer and the organic layer were neutralized and washed with saturated solution of sodiumchloride. The organic layer was dried over sodium sulfate, filtered, concentrated, dried under reduced pressure, and distilled under reduced pressure to obtain 2-butylthiophene (transparent and colorless liquid, 0.076 mol). Yield: 43%.

(4) Synthesis of 2-butyl-5-borondimethoxidethiophene as Intermediate

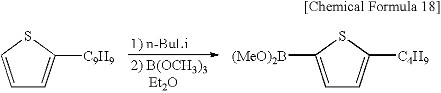

After an n-butyllithium/hexane solution (7.140 mmol) was added to a tetrahydrofuran solution of 2-butylthiophene (7.140 mmol) cooled to −50° C. and the obtained solution was stirred at about −40° C. for 3 hours, the solution was cooled to −50° C. again. Trimethyl borate (7.850 mmol) was then added to the solution, and the obtained solution was stirred at room temperature for 15 hours. 2-butyl-5-borondimethoxidethiophene (about 1.51 g) as white consistency oil obtained by removing the solvent under reduced pressure was used for the following Suzuki coupling reaction as it was.

(5) Synthesis of 1-(5′-butyl-2′-thienyl)-4-(5″-octyl-2″-thienyl)-benzene

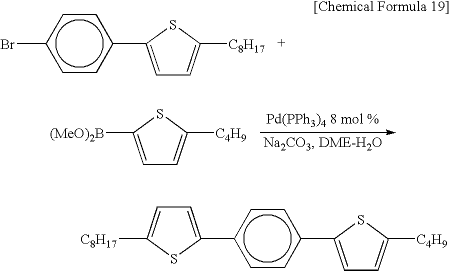

After heating a suspension of 1-bromo-4-(5′-octyl-2′-thienyl)benzene (5.980 mmol), 2-butyl-5-borondimethoxidethiophene (7.140 mmol), sodium carbonate (14.28 mmol), tetrakis(triphenylphosphine palladium) (0) (0.500 mmol), 45 mL of ethylene glycol dimethyl ether and 10 mL of water at 85° C. for about 12 hours, the suspension was ice-cooled and water was added. The suspension was then extracted with methylene chloride, and the extracted solution was washed with dilute hydrochloric acid. Then, the solution was washed with saturated solution of sodium chloride and then distilled water. The organic layer was dried over sodium sulfate, filtered, concentrated and dried under reduced pressure to obtain a crude product. This crude product was purified by column chromatography to obtain 1-(5′-butyl-2′-thienyl)-4-(5″-octyl-2″-thienyl)-benzene (4.510 mmol). Yield: 75%. This product was then recrystalized and purified by sublimation.

¹H-NMR (CDCl₃, Me₄Si) δ:

0.88 (t, J=7.1 Hz, 3H), 0.95 (t, J=7.6 Hz, 3H), 1.27-1.44 (m, 12H), 1.68 (m, 4H), 2.81 (m, 4H), 6.73 (dd, J=3.6 Hz, 2H) , 7.12 (d, J=3.4 Hz, 2H) 7.53 (s, 4H)

The analysis results confirmed that the obtained compound was the title compound. UV-Vis spectrum (chloroform solution); λₘₐₓ=342 nm (loge4.23)

### (6) Liquid Crystal Temperature Range

Differential scanning calorimetry (DSC) and polarization microscopy demonstrate that 8TPT4 transits from the isotropic phase to a highly ordered mesophaseat 141° C., and to a crystal phase at 12° C.

### (7) Charge Transport Characteristics

The charge transport characteristics of the liquid crystalline substance were measured by the TOF method. Referring to an ITO sandwich cell used for measuring, there was used a cell of which both a positive and negative electrodes were an ITO electrode; a distance between the electrodes was 16.5 μm; and an electrode area was 0.25 cm². The liquid crystalline substance was encapsulated in the cell under a condition of a temperature of 155° C., and the cell was used as a TOF measurement sample cell. The measurement was conducted at an irradiation wavelength of 337 nm at each of temperatures of 120° C., 100° C., 80° C., 60° C., 40° C. and 27° C.

The holetransport took place at each of the temperatures, and the charge mobility did not depend on field intensity at any temperature. A high hole mobility was 3×10⁻² cm²/Vs.

### Example 3

### 1-(5′-Dodecyl-2′-thienyl)-4-(5″-octyl-2″-thienyl)-benzene (8TPT12)

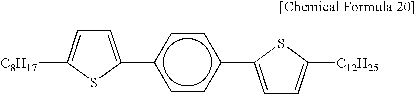

(1) Synthesis of 2-dodecylthiophene as Intermediate

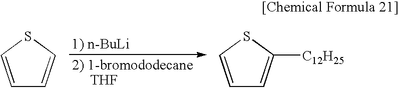

After adding an n-butyllithium/hexane solution (0.178 mol) to a tetrahydrofuran solution of thiophene (0.178 mol) cooled to −70° C., and reacting them at room temperature for 3 hours, the obtained solution was cooled to −60° C. 1-Bromododecane (0.178 mol) was then dropped into the solution to react them at room temperature for 20 hours. After removing the solvent, 200 mL of water was added into the reaction vessel ice-cooled, and the solution was extracted with 300 mL of diethyl ether. The aqueous layer was re-extracted with 200 mL of diethyl ether, and the organic layer was neutralized and washed with saturated solution of sodium chloride. The organic layer was dried over sodium sulfate, filtered, concentrated, dried under reduced pressure, and distilled under reduced pressure to obtain 2-dodecylthiophene (transparent and colorless liquid, 0.122 mol). Yield: 68%.

(2) Synthesis of 2-dodecyl-5-borondimethoxidethiophene as Intermediate

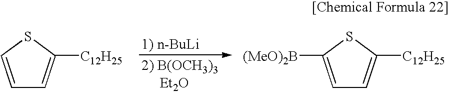

To a solution of 2-dodecylthiophene(19.81 mmol) in diethylether was added n-buthyllithium/hexane solution at −75° C. After the mixture was stirred for 3 h at r.t., the solution was cooled to −75° C. Trimethyl borate (19.81 mol) was then added to the solution, and the solution was stirred at room temperature for 15 hours. 2-dodecyl-5-borondimethoxidethiophene (about 6.4 g) as white consistency oil was obtained by removing the solvent in a reduced pressure it was used for the following Suzuki coupling reaction.

(3) Synthesis of 1-(5′-dodecyl-2′-thienyl)-4-(5″-octyl-2″-thienyl)-benzene

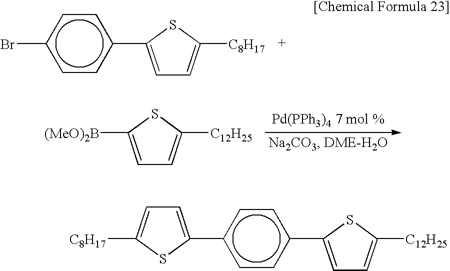

After heating a suspension of 1-bromo-4-(5′-octyl-2′-thienyl)benzene (13.21 mmol), 2-dodecyl-5-borondimethoxidethiophene (19.81 mmol), tetrakis(triphenylphosphine palladium) (0) (0.925 mmol), sodium carbonate (26.42 mmol), 70 mL of ethylene glycol dimethyl ether and 20 mL of water at 85° C. for about 7 hours, the suspension was ice-cooled and water was added thereto. Next, the suspension was extracted with 300 mL of chloroform, and the extract solution was washed with saturated solution of sodium chloride and then distilled water. The organic layer was dried over sodium sulfate, filtered, concentrated and dried under reduced pressure to obtain a crude product. This crude product was purified by column chromatography to obtain 1-(5′-dodecyl-2′-thienyl)-4-(5″-octyl-2″-thienyl)-benzene (4.207 mmol). Yield: 32%. This product was then recrystalized and purified by sublimation.

¹H-NMR (CDCl₃, Me₄Si) δ:

0.87 (t, J=7.3 Hz, 6H), 1.26-1.39 (m, 28H), 1.69 (m, 4H), 2.81 (t, J=7.8 Hz, 4H), 6.74 (d, J=3.4 Hz, 2H), 7.13 (d, J=3.7 Hz, 2H) 7.53 (s, 4H).

The analysis results confirmed that the obtained compound was the title compound. UV-Vis spectrum (chloroform solution); λₘₐₓ=341 nm (loge4.24)

### (4) Liquid Crystal Temperature Range

Differential scanning calorimetry (DSC) and polarization microscopy demonstrate that 8TPT12 transits from the isotropic phase to a mesophase having a high orientation order at 136° C. and to another mesophase having a higher orientation order at 44° C.

### (5) Charge Transport Characteristics

The charge transport characteristics of the liquid crystalline substance were measured by the TOF method. Referring to an ITO sandwich cell used for measuring, there was used a cell of which both a positive and negative electrodes were an ITO electrode; a distance between the electrodes was 12.1 μm; and an electrode area was 0.25 cm². The liquid crystalline substance was encapsulated in the cell under a condition of a temperature of 150° C., and the cell was used as a TOF measurement sample cell. The measurement was conducted at 337 nm at 120° C., 100° C., 80° C., 60° C., 40° C. and 27° C.

The hole transport took place at 120° C., and the charge mobility did not depend on field intensity. The hole mobility was 2×10⁻² cm²/Vs. At 100° C., the hole mobility was 3×10⁻² cm²/Vs; at 80° C., the hole mobility was 4×10⁻² cm²/Vs; at 60° C., the hole mobility was 6×10⁻² cm²/Vs; and furthermore, at 40° C. and 27° C., the hole mobility was 7×10⁻² cm²/Vs.

### Example 4

### 1,4-Bis(5′-dodecyl-2′-thienyl)-benzene (12TPT12)

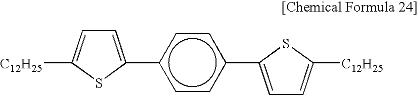

(1) Synthesis of 1,4-Bis(5′-dodecyl-2′-thienyl)-benzene

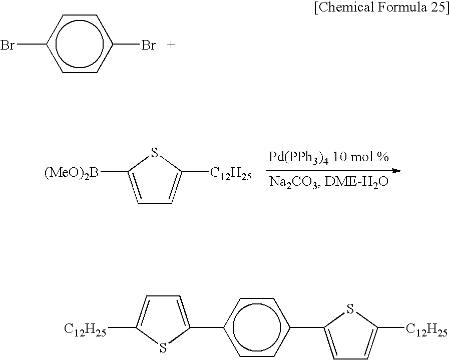

After heating a suspension of 1,4-dibromobenzene (4.728 mmol), 2-dodecyl-5-borondimethoxidethiophene (14.18 mmol), tetrakis(triphenylphosphine palladium) (0) (0.473 mmol), sodium carbonate (9.456 mmol), 34 mL of ethylene glycol dimethyl ether and 14 mL of water at 85° C. for about 5 hours, the suspension was ice-cooled and water was added thereto. Next, the suspension was extracted with 300 mL of chloroform, and the extract solution was washed with saturated solution of sodium chloride and then distilled water. The organic layer was dried over sodium sulfate, filtered, concentrated and dried under reduced pressure to obtain 1,4-bis(5′-dodecyl-2′-thienyl)-benzene (0.946 mmol) . Yield: 20%. This crude product was purified by column Chromatography, recrystalized and purified by sublimation.

¹H-NMR (CDCl₃, Me₄Si) δ:

0.87 (t, J=7.1 Hz, 6H), 1.26-1.39 (m, 36H), 1.68 (m, 4H), 2.81 (t, J=7.8 Hz, 4H), 6.74 (d, J=3.6 Hz, 2H), 7.13 (d, J=3.4 Hz, 2H) 7.53 (s, 4H).

The analysis results confirmed that the obtained compound was the title compound. UV-Vis spectrum (chloroform solution); λₘₐₓ=342 nm

### (2) Liquid Crystal Temperature Range

Differential scanning calorimetry (DSC) and polarization microscopy demonstrate that 12TPT12 transits from the isotropic phase to a mesophase at 133° C., to a mesophase having a high orientation order at 124° C., to a mesophase having a higher orientation order at 85° C., and further to a crystal phase at 67° C.

### (3) Charge Transport Characteristics

The charge transport characteristics of the liquid crystalline substance were measured by the TOF method. Referring to an ITO sandwich cell used for measuring, there was used a cell of which both a positive and negative electrodes were an ITO electrode; a distance between the electrodes was 16.8 μm; and an electrode area was 0.25 cm². The 12TPT12 was encapsulated in the cell under a condition of a temperature of 140° C., and the cell was used as a TOF measurement sample cell. The measurement was conducted at an irradiation wavelength of 337 nm at 130° C., 120° C., 100° C. and 80° C.

The hole transport of the holes took place at 130° C., and the charge mobility did not depend on field intensity. The value of the hole mobility was 4×10⁻³ Cm²/Vs. At 120° C., the hole mobility was 2×10⁻² cm²/Vs; at 100° C., the hole mobility was 4×10⁻² cm²/Vs; and at 80° C., the hole mobility having a very high value of 7×10⁻² cm²/Vs was obtained.

### Example 5

### 1-(5′-Dodecyl-2′-thienyl)-4-(5″-propyl-2″-thienyl)-benzene (12TPT3)

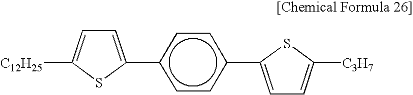

(1) Synthesis of 1-(5′-dodecyl-2′-thienyl)-4-(5″-propyl-2″ -thienyl)-benzene

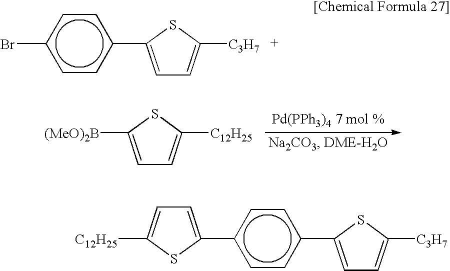

After heating a suspension of 1-bromo-4-(5′-propyl-2′-thienyl)benzene (12.89 mmol), 2-dodecyl-5-borondimethoxidethiophene (14.18 mmol), tetrakis(triphenylphosphine palladiums) (0) (0.902 mmol), sodium carbonate (25.78 mmol), 36 mL of ethylene glycol dimethyl ether and 19 mL of water at 85° C. for about 7 hours, the suspension was ice-cooled and water was added thereto. Next, the suspension was extracted with 300 mL of chloroform, and the extract solution was washed with saturated solution of sodium chloride and then distilled water. The organic layer was dried over sodium sulfate, filtered, concentrated and dried under reduced pressure to obtain a crude product. This crude product was purified by column Chromatography to obtain 1-(5′-dodecyl-2′-thienyl)-4-(5″-propyl-2″-thienyl)-benzene (1.193 mmol). Yield: 20%. This product was then recrystalized and purified by sublimation.

¹H-NMR (CDCl₃, Me₄Si) δ:

0.87 (t, J=7.1 Hz, 3H), 1.00 (t, J=7.3 Hz, 3H), 1.26-1.38 (m, 18H), 1.65-1.77 (m, 4H), 2.77-2.83 (m, 4H), 6.74 (dd, 2H), 7.12 (d, J=1.5 Hz, 1H), 7.13 (d, J=1.5 Hz, 1H) , 7.53 (s, 4H)

The analysis results confirmed that the obtained compound was the title compound. UV-Vis spectrum (chloroform solution); λₘₐₓ=342 nm

### (2) Liquid Crystal Temperature Range

Differential scanning calorimetry (DSC) and polarization microscopy demonstrate that 12TPT3 transits from the isotropic phase to a mesophase at 134° C. and to a crystal phase at 64° C.

### Example 6

### 1-(5′-Dodecyl-2′-thienyl)-4-(2″-thienyl)-benzene (8TPT)

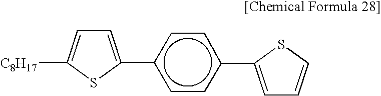

(1) Synthesis of 1-(5′-dodecyl-2′-thienyl)-4-(2″-thienyl)-benzene (8TPT)

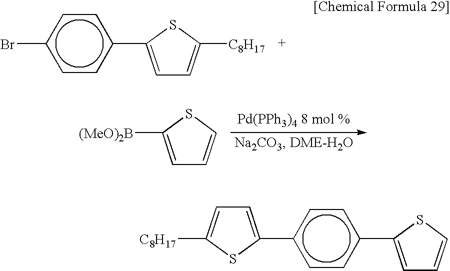

After heating a suspension of 1-bromo-4-(5′-octyl-2′-thienyl)benzene (8.425 mmol), 2-borondimethoxidethiophene (16.85 mmol), sodium carbonate (16.85 mmol), tetrakis(triphenylphosphine palladium) (0) (0.674 mmol), 30 mL of ethylene glycol dimethyl ether and 13 mL of water at 85° C. for about 4 hours, the suspension was ice-cooled and water was added thereto. The suspension was then extracted with 100 mL of chloroform, and the extract solution was washed with dilute hydrochloric acid. Then, the solution was washed with saturated solution of sodium chloride and then distilled water. The organic layer was dried over sodium sulfate, filtered, concentrated and dried under reduced pressure to obtain a crude product. This crude product was purified by column Chromatography to obtain 1-(5′-dodecyl-2′-thienyl)-4-(2″-thienyl)-benzene (5.584 mmol). Yield: 66%. This product was then recrystalized.

¹H-NMR (CDCl₃, Me₄Si) δ:

0.88 (t, J=6.6 Hz, 3H), 1.28-1.38 (m, 10H), 1.70 (m, 2H), 2.81 (t, J=7.6 Hz, 2H), 6.75 (d, J=3.4 Hz, 1H), 7.08 (dd, J=5.1 Hz, 1H), 7.15 (d, J=3.4 Hz, 1H), 7.27 (dd, J=5.1 Hz, 1H), 7.32 (d, J=3.6 Hz, 1H), 7.54-7.60 (m, 4H)

The analysis results confirmed that the obtained compound was the title compound. UV-Vis spectrum (chloroform solution); λₘₐₓ=341 nm

### (2) Liquid Crystal Temperature Range

Differential scanning calorimetry (DSC) and polarization microscopy demonstrate that 8TPT transits from the isotropic phase to a mesophase at 135° C. and to a crystal phase at 90° C.

### Example 7

### 1-(5′-Hexyl-2′-thienyl)-4-(5″-propyl-2″-thienyl)-benzene (6TPT3)

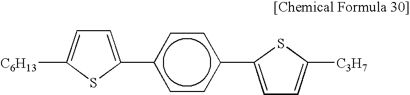

(1) Synthesis of 1-(5′-hexyl-2′-thienyl)-4-(5″-propyl-2″-thienyl)-benzene

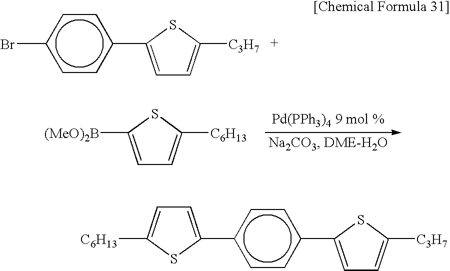

After heating a suspension of 1-bromo-4-(5′-propyl-2′-thienyl)benzene (12.02 mmol), 2-hexyl-5-borondimethoxidethiophene (12.02 mmol), tetrakis(triphenylphosphine palladium) (0) (1.030 mmol), sodium carbonate (24.04 mmol), 30 mL of ethylene glycol dimethyl ether and 16 mL of water at 85° C. for about 5 hours, the suspension was ice-cooled and water was added thereto. Next, the suspension was extracted with 300 mL of chloroform, and the extract solution was washed with saturated solution of sodium chloride and then distilled water. The organic layer was dried over sodium sulfate, filtered, concentrated and dried under reduced pressure to obtain a crude product. This crude product was purified by column chromathography to obtain 1-(5′-hexyl-2′-thienyl)-4-(5″-propyl-2″-thienyl)-benzene (10.38 mmol) Yield: 87%. This product was then recrystalized and purified by sublimation.

¹H-NMR (CDCl₃, Me₄Si) δ:

0.87 (t, J=7.3 Hz, 3H), 1.00 (t, J=7.3 Hz, 3H) 1.26-1.43 (m, 6H), 1.65-1.77 (m, 4H), 2.77-2.83 (q, J=6.8 Hz, 4H), 6.74 (m, 2H), 7.12 (d, J=1.5 Hz, 1H), 7.13 (d, J=1.2 Hz, 1H), 7.53 (s, 4H).

The analysis results confirmed that the obtained compound was the title compound. UV-Vis spectrum (chloroform solution); λₘₐₓ=341 nm

### (2) Liquid Crystal Temperature Range

Differential scanning calorimetry (DSC) measurement and polarization microscopy demonstrate that 6TPT3 transits from the isotropic phase to a mesophase at 147° C. and to a crystal phase at 45° C.

### Example 8

### 1-(5′-Pentynyl-2′-thienyl)-4-(5″-propyl-2″-thienyl)-benzene (3TPTyne3)

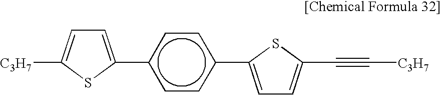

(1) Synthesis of 1-(5′-pentynyl-2′-thienyl)-4-(5″-propyl-2″-thienyl)-benzene

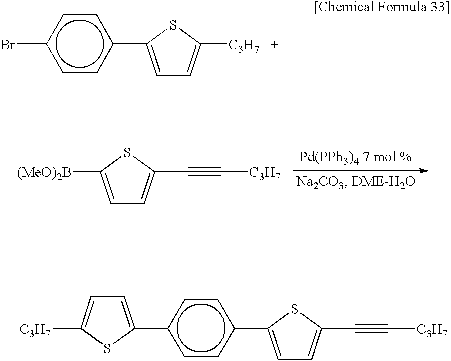

After heating a suspension of 1-bromo-4-(5′-propyl-2′-thienyl)benzene (10.13 mmol), 2-pentynyl-5-borondimethoxidethiophene (20.26 mmol), tetrakis(triphenylphosphine palladium) (0) (0.709 mmol), sodium carbonate (20.26 mmol), 43 mL of ethylene glycol dimethyl ether and 15 mL of water at 85° C. for about 6 hours, the suspension was ice-cooled and water was added thereto. The suspension was then extracted with 300 mL of chloroform, and the extract solution was washed with saturated solution of sodium chloride and then distilled water. The organic layer was dried over sodium sulfate, filtered, concentrated and dried under reduced pressure to obtain a crude product. This crude product was purified by column chlomatography to obtain 1-(5′-pentynyl-2′-thienyl)-4-(5″-propyl-2″-thienyl)-benzene (4.356 mmol). Yield: 43%. This product was then recrystalized and purified by sublimation.

¹H-NMR (CDCl₃, Me₄Si) δ:

1.00 (t, J=7.3 Hz, 3H), 1.05 (t, J=7.3 Hz, 3H), 1.59-1.78 (m, 4H), 2.43 (t, J=7.1 Hz, 2H), 2.80 (t, J=7.3 Hz, 2H), 6.75 (d, J=3.7 Hz, 1H), 7.07 (d, J=3.9 Hz, 1H), 7.14 (d, J=3.7 Hz, 2H), 7.53 (s, 4H).

The analysis results confirmed that the obtained compound was the title compound. UV-Vis spectrum (chloroform solution); λₘₐₓ=356 nm

1. 1,4-Dithienylbenzene derivatives represented by the following general formula (I): 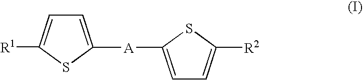
wherein R¹ and R² each independently represent a hydrogen atom or a hydrocarbyl group having 1 to 20 carbon atoms, provided that R¹ and R² are not simultaneously a hydrogen atom; and A represents an optionally substituted benzene ring.

2. The 1,4-dithienylbenzene derivatives according to claim 1, wherein A is a benzene ring.

3. The 1,4-dithienylbenzene derivatives according to claim 1 or 2, wherein R¹ and R² each independently represent a straight-chain hydrocarbyl group having 4 to 20 carbon atoms.

4. Liquid crystal compositions comprising the 1,4-dithienylbenzene derivatives according to any one of claims 1 to 3.

5. Charge transport materials comprising the 1,4-dithienylbenzene derivatives according to any one of claims 1 to 3 or liquid crystal compositions containing said derivative.

6. The charge transport materials according to claim 5, wherein the charge transport materials have a hole or electron mobility of 1×10⁻³ cm²/Vs or more.

7. A photoelectron conversion device comprising the charge transport materials according to claim 5 or 6.

8. An electroluminescent device comprising the charge transport materials according to claim 5 or 6.
